(19) United States
(12) Plant Patent Application Publication
(10) Pub. No.: US 2017/0265363 P1
(43) Pub. Date: Sep. 14, 2017

(54) Pelargonium plant named 'KLEPS15380'

(50) ;

(71) Applicant: Klemm+Sohn GmbH & Co.KG, Stuttgart (DE)

(72) Inventors: Nils Klemm, Stuttgart (DE); Martin Glawe, Stuttgart (DE)

(73) Assignee: Klemm+Sohn GmbH & Co.KG, Stuttgart (DE)

(21) Appl. No.: 14/998,954

(22) Filed: Mar. 9, 2016

(51) Int. Cl. A01H 5/02

(52) U.S. Cl. PLT/330

(57) ABSTRACT

A new pelargonium plant particularly distinguished by dark red flowers and an upright growth habit, is disclosed.

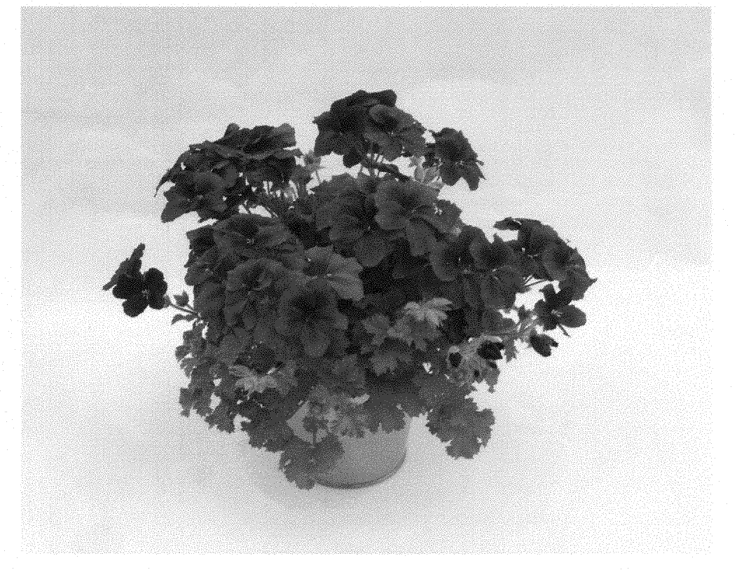

Genus and species: Pelargonium grandiflorum.

Variety denomination: ‘KLEPS15380’.

### BACKGROUND OF THE NEW PLANT

The present invention comprises a new and distinct variety of pelargonium, botanically known as Pelargonium grandiflorum and hereinafter referred to by the variety name ‘KLEPS15380’. ‘KLEPS15380’ originated from a cross-combination in August 2010 in Stuttgart, Germany between the female pelargonium variety ‘PSH-2009-C-Dark Red’ (unpatented) and the proprietary male pelargonium variety ‘PS-2006-0001” (unpatented).

Seeds from the cross-combination were obtained and sown and plants were grown for evaluation. A single plant was selected in June 2011 and subsequently named ‘KLEPS15380’. In August 2011 ‘KLEPS15380’ was first vegetatively propagated in Stuttgart, Germany by vegetative cuttings from stems and branchings. ‘KLEPS15380’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. A dark red flower color; and

2. An upright growth habit.

### DESCRIPTION OF THE PHOTOGRAPH

This new pelargonium plant is illustrated by the accompanying photograph which shows an overall view of the plant.

The photograph is of an approximately ten-week-old plant grown in Stuttgart, Germany under greenhouse conditions in April 2014. The colors shown are as true as can be reasonably obtained by conventional photographic procedures.

### DESCRIPTION OF THE NEW VARIETY

The following detailed descriptions set forth the distinctive characteristics of ‘KLEPS15380’. The data which define these characteristics were collected from asexual reproductions carried out in Stuttgart, Germany. Data was collected on plants grown 5-months after planting in Stuttgart, Germany under a glass covered greenhouse in July 2015. Color references are to The R.H.S. Colour Chart of The Royal Horticultural Society of London (R.H.S.), 5^{th} edition (2007).
Classification:
   Family.—Geraniaceae.
   Botanical.—Pelargonium grandiflorum.
   Common.—Pelargonium.
   Designation.—‘KLEPS15380’.
Parentage:
   Female parent.—The female pelargonium variety ‘PSH-2009-C-Dark Red’ (unpatented).
   Male parent.—The proprietary male pelargonium variety ‘PS-2006-0001” (unpatented).
Plant:
   Form.—Bushy and round.
   Habit.—Upright.
   Height.—22.0 cm.
   Spread.—22.0 cm.
   Natural flowering season.—Spring to autumn.
   Propagation type.—Vegetative cuttings.
   Time to produce a finished flowering plant.—4 to 5 months.
   Time to initiate and develop roots.—28 days.
   Root description.—White and fine (RHS N155C).
Stems:
   Quantity of stems per plant.—5 to 6.
   Color.—RHS 137A.
   Surface texture.—Moderately pubescent.
   Stem form.—Round.
   Diameter.—0.6 cm.
   Length.—5.0 cm to 8.0 cm.
   Internode length.—1.0 cm to 1.5 cm.
   Anthocyanin.—Absent.
Leaves:
   Arrangement.—Alternate.
   Shape.—Cordate.
   Apex.—Acute.
   Base.—Open.
   Margin.—Crenate.
   Surface texture.—Upper surface: Rough, sparsely pubescent Lower surface: Smooth.
   Length.—3.0 cm to 4.0 cm.
   Width.—4.0 cm to 5.0 cm.
   Color.—Upper surface: RHS 137B Lower surface: RHS 137D.
   Petiole.—Length: 4.0 cm to 8.0 cm Diameter: 0.2 cm Color: RHS 137A Strength: Medium.
   Venation pattern.—Palmate.
   Venation color.—Upper surface: RHS 137D Lower surface: RHS 138B.
Inflorescence:
   Lastingness of flowers on the plant.—6 to 10 days.
   Umbels.—Quantity of umbels per plant: 5 Length: 4.0 cm to 5.0 cm Diameter: 5.0 cm to 6.0 cm.
   Individual florets of the umbel.—Quantity of florets per umbel: 4 to 5 Depth: 2.0 cm Diameter: 4.0 cm.
   Fragrance.—Absent in flower.
Flower buds:
   Cluster shape.—Round.
   Cluster size.—Length: 1.0 cm to 1.5 cm Width: 1.0 cm to 1.5 cm.
   Surface texture.—Moderately pubescent.
   Bud.—Height: 0.8 cm to 1.0 cm Width: 0.5 cm Shape: Elliptic Apex: Acute Base: Obtuse Margin: Smooth Color: RHS 137D.
Petals:
   Quantity per flower.—6.
   Shape.—Drop-shaped.
   Apex.—Entire.
   Base.—Obtuse.
   Margin.—Entire.
   Length.—3.0 cm.
   Width.—2.5 cm.
   Surface texture.—Upper surface: Smooth Lower surface: Smooth.
   Color of lobes.—Upper lobes: Upper surface: RHS N79A Lower surface: RHS N79B Lower lobes: Upper surface: RHS N79A Lower surface: RHS N79A.
Sepals:
   Number per flower.—5.
   Shape.—V-shaped.
   Apex.—Cuspidate.
   Base.—Obtuse.
   Margin.—Entire.
   Surface texture.—Upper surface: Smooth Lower surface: Moderately pubescent.
   Color.—Upper surface: RHS 137D Lower surface: RHS 137D.
   Length.—1.0 cm.
   Width.—0.4 cm.
Pedicel:
   Length.—2.0 cm.
   Diameter.—0.1 cm.
   Color.—RHS 137C.
   Surface texture.—Moderately pubescent.
   Strength.—Medium.
Peduncle:
   Length.—5.0 cm to 7.0 cm.
   Diameter.—0.2 cm.
   Color.—RHS 137C.
   Surface texture.—Moderately pubescent.
   Strength.—Medium.
Reproductive organs:
   Stamens.—Quantity of stamens per flower: 4 to 7 Anther length: 0.2 cm Filament length: 0.8 cm Filament color: RHS 72C Pollen amount: Moderate Pollen color: RHS 72D.
   Pistils.—Number: 1 Stigma color: RHS N79C Style length: 1.0 cm Style color: RHS 72C Ovary arrangement: Central Ovary color: RHS 138D Ovary diameter: 0.1 cm Ovary length: 0.1 cm.
Fruit and seed set: None observed.
Disease and pest/insect resistance: None observed.
Comparison with parental lines and known variety: ‘KLEPS15380’ is distinguished from its parents as described in Table 1:

TABLE 1
Comparison with Parental Lines

| | | Female parent | |
|---|---|---|---|
| | | ‘PSH-2009-C- | Male Parent |
| Characteristic | ‘KLEPS15380’ | Dark Red’ | ‘PS-2006-0001” |
| Flower size | Medium | Medium-big | Very small |
| Flower color | Dark red | Purple-red with a | Purple with a |
| | | dark red eye | dark purple eye |

‘KLEPS15380’ can be compared to the commercial variety CANDY FLOWERS Dark Red, also known as ‘Camdared’ (U.S. Plant Pat. No. 20,418). Differences between the two varieties are described in Table 2:

TABLE 2
Comparison with Similar Variety

| | | Commercial line |
|---|---|---|
| | | CANDY FLOWERS |
| Characteristic | ‘KLEPS15380’ | Dark Red |
| Flower color | Dark red | Dark red and purple |
| | | bi-colored flowers |
| Leaves | More crenated than | Less crenated than |
| | CANDY FLOWERS | ‘KLEPS 15380’ |
| | Dark Red | |

We claim:

1. A new and distinct variety of pelargonium plant named ‘KLEPS15380’ as illustrated and described herein.
